(19) United States
(12) Patent Application Publication
(10) Pub. No.: US 2017/0010252 A1
(43) Pub. Date: Jan. 12, 2017

(54) Unseen Benefit Indicator System

(71) Applicant: The Procter & Gamble Company, Cincinnati, OH (US)

(72) Inventors: Delia Bearup, Monroe, OH (US); Chelsay Lynn Brewster, Hamilton, OH (US); Karen Chan, Dayton, OH (US); Tinlee Lin, Cincinnati, OH (US)

(21) Appl. No.: 15/185,047

(22) Filed: Jun. 17, 2016

Related U.S. Application Data
(60) Provisional application No. 62/189,115, filed on Jul. 6, 2015.

(51) Int. Cl. G01N 33/483; G01N 33/497; G06Q 30/02; G01N 33/487

(52) U.S. Cl. CPC G01N 33/483; G01N 33/487; G01N 33/497; G06Q 30/0269

(57) ABSTRACT

An unseen benefit indicator kit containing a vitamin, mineral, supplement product, a first biomarker test, and a final biomarker test. The kit provides an unseen benefit indicator that allows users to see health benefits while taking the vitamin, mineral, supplement product.

### FIELD OF THE INVENTION

The invention relates to an unseen benefit indication system, particularly a means for users to consumers to see health benefits while taking vitamin, mineral, supplement products.

### BACKGROUND OF THE INVENTION

Many consumers are interested in improving their health and wellbeing and take vitamins, minerals, and supplement (VMS) products to improve their health. While some people claim to feel a benefit, such as improved energy or reduced stress, soon after starting to take VMS products, many consumers have no idea if the product is providing any benefit whatsoever. This is especially true with products that are being taken for systemic health, such as supporting or improving heart health and bone health, reducing inflammation, and/or supporting one's immune system.

Since consumers cannot see or feel the benefit of some VMS products, it is difficult for them to know whether or not the product is actually providing a benefit. Therefore, many consumers are unlikely to continue using the VMS product or use the product sporadically, even if it is benefiting them.

Thus, there is a need for an unseen benefit indicator that allows consumers to see health benefits while taking VMS products.

### SUMMARY OF THE INVENTION

An unseen benefit indicator kit comprising: (a) a vitamin, mineral, supplement product; (b) a first biomarker test or a voucher for a first biomarker test or a coupon for a first biomarker test; (c) a final biomarker test or a voucher for a final biomarker test or a coupon for a final biomarker test; wherein the kit provides an indicator system that allows a user to see a health benefit while taking the vitamin, mineral, supplement product.

A method for seeing an unseen benefit comprising: (a) taking a first biomarker test and receiving a first biomarker test result; (b) consuming a vitamin, mineral, supplement product; (c) taking a final biomarker test and receiving a final biomarker test result; comparing the first biomarker test result and the final biomarker test result to see an unseen benefit.

### BRIEF DESCRIPTION OF THE DRAWINGS

FIG. 1 shows the results of an unseen benefit indicator where consumers took fish oil supplements with and without receiving health metrics; and

FIG. 2 shows the results of an unseen benefit indicator of a subset of data in FIG. 1 where consumers took fish oil supplements and received health metrics.

FIG. 3 shows the results of an unseen benefit indicator where consumers took fiber supplements with and without receiving health metrics; and

FIG. 4 shows the results of an unseen benefit indicator of a subset of data in FIG. 1 where consumers took fiber supplements and received health metrics.

### DETAILED DESCRIPTION OF THE INVENTION

It can be difficult for consumers to feel or see any changes in their health when they begin using VMS products. This means that consumers, who initially started a VMS regimen, frequently do not continue their regimen or do so irregularly, potentially negating the long-term benefits. However, the VMS product may have the intended effect and be improving health markers, but the consumer cannot see these benefits. Therefore an unseen benefit indicator that that allows consumers to see proof of health benefits of VMS products is desirable.

One way for consumers to be able to see the changes from a VMS product is to use the unseen benefit indicator as described herein. The unseen benefit indicator can include at least a baseline reading of one or more biomarkers, one or more VMS products, which generally includes about two to four weeks of doses, and a final health assessment that will re-test one or more biomarkers.

As used herein, “VMS” or “vitamins, minerals, and supplements” refers to vitamins, minerals, fiber, fatty acids, amino acids and proteins, herbals, probiotics, hormones and combinations thereof. VMS products can include one or more vitamins, minerals, and/or supplements, which can be combined with excipients.

As used herein, the term “non-GMO” means that the product has been verified as fully compliant to the Non-GMO Project Standard. Meets the standards of the non-GMO Project Standard as of May 21, 2014, and thus the product can be labeled Non-GMO Project Verified. As used herein, the term “biomarker” refers to any biological molecules (for instance, genes, proteins, lipids, metabolites) that, singularly or collectively, reflect the current or predict future physiological state.

In one example, twenty-nine (29) consumers were selected to begin a fourteen (14) day regimen of New Chapter® Wholemega™ Whole Fish Oil. The consumers who participated were interested in improving their heart health markers and were also interested in purchasing Wholemega™ fish oil based on a product concept shown to them, but were not currently taking a fish oil supplement regularly. The consumers were given a fourteen (14) day supply of Wholemega™ and two (2) vouchers for a quick lipids panel at a healthcare clinic. They visited the healthcare clinic before they started using the fish oil for a baseline lipid panel and then after fourteen (14) days of usage. Soon after the second lipid panel, a pharmacist walked the consumers through their tests and explained any changes. Then, the consumers went to a central site to discuss their experience and they were contacted after two additional weeks, to see how, if at all, their habits had changed given their participation in the study. In this example, the lipid panel focused on triglycerides and consumers were hoping to improve heart health by lowering triglycerides.

Previously a similar study was conducted where 130 consumers were selected to begin a 30 day regimen of New Chapter® Wholemega™ Whole Fish Oil. The consumers who participated were interested in purchasing Wholemega™ based on a product concept shown to them. The consumers were given a thirty (30) day supply of Wholemega™. They were asked a series of questions via an online questionnaire before they used the product and after thirty (30) days of usage. The questionnaire included questions about purchase interest and how well the product worked for metrics such as heart health. Consumers were not given any information about their own health metrics during this previous study.

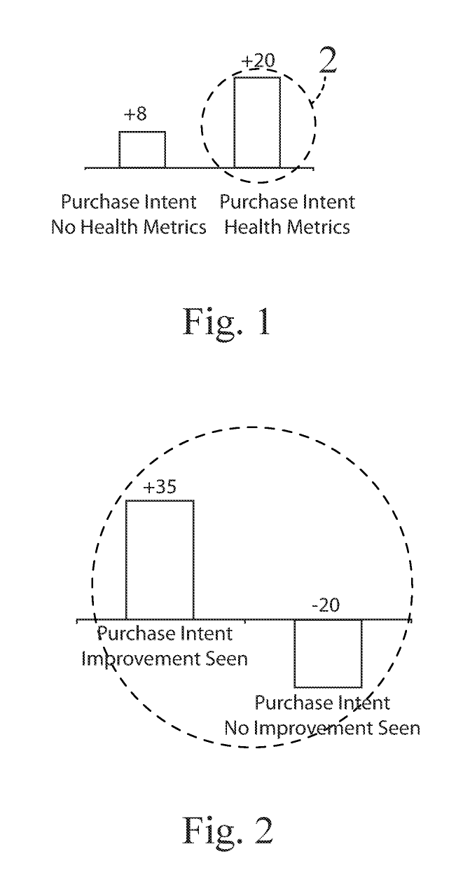

FIG. 1 shows the differences between the two examples. In the example where the consumers did not receive health metrics, their intent to repurchase increased 8% from initial concept to after two weeks of use. However, when they received health metrics the purchase intent increased 20% from initial concept to after two weeks of use. As shown in FIG. 2, in the group that received health metrics, those that claimed they saw an improvement in their health metrics had an increase in purchase intent of 35% from initial concept. However, those who did not see an improvement had a 20% decrease in their purchase intent. When the consumers who received biomarkers were contacted two or three weeks after their use, 14% of had bought a fish oil product, about half had recommended the product to a family member or friend, and about 20% of them had spent time talking to their doctor about their heart health.

In addition, the consumers could be given additional information about the fish oil product they were consuming For instance, if the consumers were given Wholemega™ Whole Fish Oil, they could also be given information about the extra-virgin process for making the extract, the color and transparency of the supplement product, the sourcing of the Alaskan Salmon, human clinical trials, the ingredients in the fish oil including the omega fatty acid profile, non-GMO Project Standard, and other information. This information could be included in the packaging and marketing materials and/or provided by a healthcare professional or someone who is knowledgeable about supplements.

In one example, thirty-eight (38) consumers were selected to begin a fifty-six (56) day regimen of the fiber supplement Meta Daily Heart Health Berry (sold by The Procter & Gamble Company, Cincinnati, Ohio). The consumers who participated were interested in managing their cholesterol levels and were also interested in purchasing Meta Daily Heart Health based on a product concept shown to them. The consumers were given a fifty-six (56) day supply of Meta Daily Heart Health Berry and two (2) vouchers for a quick lipids panel at a healthcare clinic. They visited the healthcare clinic before they started using the fiber supplement for a baseline lipid panel and then after fifty-six (56) days of usage. Soon after the second lipid panel, a pharmacist walked the consumers through their tests and explained any changes. They were asked a series of questions via an online questionnaire before they used the product and after fifty-six (56) days of usage. The questionnaire included questions about purchase interest and how well the product worked for metrics such as heart health. In this example, the lipid panel focused on total cholesterol and LDL cholesterol.

In the same study, a second leg of thirty-eight (38) consumers were selected to begin a fifty-six (56) day regimen of the fiber supplement Meta Daily Heart Health Berry. The consumers who participated were interested in managing their cholesterol levels and were also interested in purchasing Meta Daily Heart Health based on a product concept shown to them. The consumers were given a fifty-six (56) day supply of Meta Daily Heart Health Berry. They were asked a series of questions via an online questionnaire before they used the product and after fifty-six (56) days of usage. The questionnaire included questions about purchase interest and how well the product worked for metrics such as heart health. Consumers were not given any information about their own health metrics during this leg of the study.

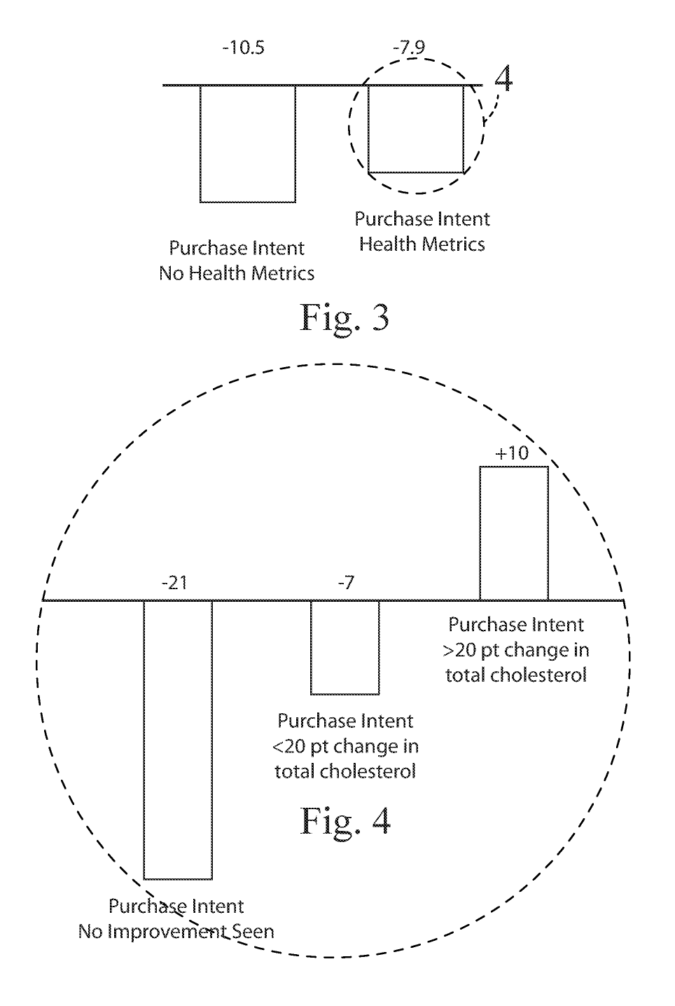

FIG. 3 shows the differences between the two examples. In the example where the consumers did not receive health metrics, their intent to repurchase decreased 11% from initial concept to after eight weeks of use. However, when they received health metrics the purchase intent decreased only about 8% from initial concept to after eight weeks of use. As shown in FIG. 4, in the group that received health metrics, those that saw no improvement in their health metrics or saw less than a 20 point improvement in total cholesterol had a decrease in purchase intent of 21% and 7% from initial concept, respectively. However, those who saw a greater than 20 point improvement in total cholesterol had a 10% increase in their purchase intent. In this study it was found that a greater than 20 point change in total cholesterol is needed to drive consumer purchase intent.

In another example, the methodology described above could be used for people, 12 years and older, who are interested in taking a fiber supplement, in particular a psyllium fiber such as Metamucil® (sold by The Procter & Gamble Company, Cincinnati, Ohio). Consumers could be selected if they have an interest in promoting heart health, digestive health, maintaining healthy blood sugar, and/or feeling less hungry between meals and if they are also interested in taking a psyllium fiber supplement, but do not currently take a psyllium fiber supplement or in some examples any fiber supplement. The consumers could be given a twenty-eight (28) day supply of psyllium fiber, instructions for use, and two vouchers or tests. If the consumer is taking the psyllium fiber to feel less hungry between meals, then the directions will instruct her to take two round teaspoons in 8 or more ounces (oz) of liquid up to three (3) times daily. If the consumer is taking the psyllium fiber to lower her cholesterol, then the consumer can take one rounded teaspoon in 8 oz of water, three (3) times daily. If the consumer is taking the psyllium for maintaining healthy blood sugar levels as part of her diet, then the directions will instruct her to take one rounded teaspoon in 8 oz of liquid, three (3) times daily. For any of these regiments, in particular the regimen for consumers who are interested in maintaining healthy blood sugar levels and/or consumers who are taking medication, it may be advisable for them to consult with a health care practitioner, in particular a doctor or other primary care provider to discuss the regimen. The test can measure one or more health markers. For instance, the test can be a quick lipid panel and can measure triglycerides, high-density lipoprotein (HDL) cholesterol, low-density lipoprotein (LDL) cholesterol, total cholesterol and/or blood sugar level. The consumer can be given the results and can review the results with a healthcare professional such as a nurse, nurse practitioner, physician's assistant, doctor, pharmacist, and/or technicians. After the second test, if the consumer intends to repurchase psyllium, in some instances she can be given a coupon to buy psyllium fiber, in particular a certain brand of psyllium fiber. She could also be given coupons for other products that contain psyllium, such as wafers, snack bars, capsules, and/or other product forms. In another example, the cost of the VMS product in the kit and/or the one or more biomarker tests can also be reduced.

In another example, the unseen benefit indicator can be used to determine whether or not a VMS product provides a whole-body healthy inflammation response and possibly reduce inflammation. One example of a product like this is Zyflamend™ Whole Body (available from New Chapter®), which is an herbal supplement that can contain rosemary, turmeric, ginger, holy basil, organic green tea, hu zhang, Chinese goldthread, barberry, organic oregano, and/or Chinese skullcap. The Zyflammend™ supplement can be taken twice a day with food. A person who is looking to improve their inflammation response could purchase a kit that contains a two week supply of Zyflammend™ (24 softgels) and at least two vouchers or coupons for a biomarker test. Many people may be looking to improve their inflammation response and could include athletes or people who participate in a lot of physical activity, people with stiff joints, and others. Before beginning the regimen, a consumer would take a biomarker test. The biomarker test could measure one or more inflammation markers selected from the group consisting of NFkB, PGE2, COX1, COX2, NRF-2 (ARE), LTB4, SLOX, 12LOX, CRP, Interleukins (e.g. IL-1, IL-6), TNFα, INF and combinations thereof. After taking the supplement for two weeks, the consumer would get another biomarker test. She could then compare the results, possibly with a healthcare professional. A change in one or more of the biomarkers could indicate that the supplement is helping to control inflammation. If the consumer felt different and/or had changes in their inflammation biomarkers, then the consumer could continue the regimen with the supplement they were taking, for instance Zyflammend™ Whole Body. However, if the results were insignificant or the consumer was otherwise displeased, the biomarker results could be analyzed and a different VMS product and/or combination of VMS products could be recommended.

In another example, the unseen benefit indicator could be used to determine whether or not a probiotic product is effective. It can be especially difficult for a consumer to tell whether a probiotic product is effective because the changes occur over time and/or with gastrointestinal problems, consumers generally do not have daily episodes. Therefore, a baseline can be established using a biomarker test and/or by having a consumer track their gastrointestinal health over a period of time, for instance two weeks or a month. Then the consumer can start taking the probiotic regimen. In some instances, the consumer will continue to track their gastrointestinal health daily. After completing the regimen, consumers may take a final biomarker test. They can analyze their results, either biomarkers and/or daily logs, to determine whether or not the probiotic has had an effect. In one example, the daily logs will ask questions about abdominal pain/discomfort, severity of bloating/distention, etc. In another example, the biomarkers will measure inflammation. If the probiotic works, the consumer can continue taking. If the probiotic does not work, then another treatment may be recommended. This could include a different strain of probiotics or possibly a fiber supplement. In one example, the consumer will start a regimen that includes both a probiotic and a fiber supplement, for instance a psyllium fiber supplement.

In another example, the unseen benefit indicator could be used to determine carbohydrate status in people trying to lose weight from a low-carbohydrate diet (ketogenic). First, they will get a baseline biomarker test, which will measure a biomarker related to weight loss. Then they will begin the VMS product, for instance psyllium and/or a multivitamin and/or herbal that promotes health weight and/or satiety. After a period of time, they can get another biomarker test. In one example, the biomarker test can measure ketones in sweat, saliva, and/or breath. If ketones are elevated, then it could be an indication that the user is burning fat. The second biomarker test can be taken a week or two after starting the regimen. However, in some instances consumers may want to wait a two, three, four, or five days and then take the ketone biomarker test. If there is a change in the baseline ketone level, it could provide an indication to consumers that they are burning fat stores.

In another example, the unseen benefit indicator can be in an advertisement. The advertisement can be a print advertisement or a video advertisement. The advertisement can show the consumer's reaction when they get their baseline biomarker results and/or the final biomarker results. In one example, the consumer may be surprised at the change in her biomarker results. In another example, the consumer could express her intention of whether or not she intends to buy the product again. The advertisement can be printed and can be a combination of words and pictures that can show the user's intent. In another example, the advertisement can be a video. The video can be available streaming over the internet and/or the television. The advertisement can show one consumer or many consumers giving their real time reactions to the VMS product, their biomarker results, or their future intentions about buying the product and/or recommending to friends and family.

In one example, from about 1 out of 10 to about 9 out of 10 users see a beneficial change in their biomarkers after two weeks, in another example from about 1 out of 5 to about 4 out of 5 users see a beneficial change, in another example from about 1 out of 4 to about 3 out of 5, and in another example from about 1 out of 3 to about 1 out of 2. In one example, the beneficial change can be lowering triglycerides after about two weeks.

In another example from about 10% to about 90% of consumers gained confidence in the VMS product by seeing the beneficial change in their biomarkers, in another example from about 20% to about 80%, in another example from about 30% to about 70%, in another example from about 40% to about 60%, and in another example from about 45% to about 55%.

In another example from about 10% to about 95% of consumers saw a beneficial change in the biomarker after taking the VMS product, in another example from about 25% to about 85%, in another example from about 35% to about 75%, in another example from about 45% to about 65%, and in another example from about 50% to about 60%.

In one example, the unseen benefit indicator can be an unseen benefit indicator kit. In some instances, the consumer can decide that she wants to start a vitamin regimen to achieve one or more health benefits. She can purchase an unseen benefit indicator kit that contains a VMS product and at least a first and a final biomarker test. The first biomarker test can be used to get a baseline of at least one biomarker. The final biomarker test can be used to retest the baseline after a predetermined amount of time. The results of the final biomarker test can help the user realize the unseen benefit of a VMS product. A consumer can be surprised to see a change in biomarkers, especially when she could not feel or see the change. This can be particularly important if the consumer is trying to improve and support things like heart health and/or immune health where the conditions develop over time and can be serious or life threatening when they occur.

The kit can contain a predetermined amount of VMS product, intended to be used after the baseline biomarker test. Ideally, the amount of VMS product in the kit can be enough to impact the biomarker, while still allowing the user to see the change in the biomarker in a relatively short amount of time. For instance, in one example, the kit contains at least about a week of VMS product, in another example at least about two weeks, in another example at least about three weeks, in another example at least about four weeks, in another example at least about five weeks, in another example at least about six weeks, in another example at least about seven weeks, in another example at least about eight weeks, in another example at least about ten weeks, in another example at least about twelve weeks. In another example the kit contains less than about twelve weeks of doses of VMS product, in another example the kit contains less than about eleven weeks, in another example less than about nine weeks, in another example less than about eight weeks, in another example less than about six weeks, in another example less than about four weeks, and in another example less than about three weeks. In one example the kit contains from about 1 week to about 16 weeks of VMS product, in another example from about 2 weeks to about 15 weeks, in another example from about 3 weeks to about 14 weeks, in another example from about 4 weeks to about 12 weeks, in another example from about 6 weeks to about 10 weeks, and in another example from about 7 weeks to about 8 weeks.

In one example the VMS can be intended to be taken one time per week, in another example the VMS product can be intended to be taken once daily, in another example the VMS product can be intended to be take twice daily, in another example the VMS product can be intended to be taken three times daily, and in another example the VMS product can be taken as desired. In one example, the VMS product can be taken on an empty stomach. In another example, the VMS product can be taken with meals. In another example, the VMS product can be taken in the morning, in another example the VMS product can be taken in the evening, and in another example the VMS product can be taken at bedtime.

In some examples, the kit can contain additional items to further aid in the health and wellness of the consumer. In one example, the kit contains one or more coupons for discounted VMS products. The coupon could be for the VMS product that was in the original kit, which would help the user repurchase the product and continue the regimen. The coupon could also be for related VMS products, for instance, if the consumer was taking a VMS product for heart health, such as fish oil, the coupon could contain a coupon for another heart health product, like psyllium fiber, that can be taken in combination with the fish oil for additional benefits. The kit could also contain a coupon for a product by the same manufacturer, company, or brand that can be intended to help with a different area of health. For instance, if the consumer is taking fish oil for heart health, there could be coupons for a product intended to reduce inflammation, support joint health, support gastrointestinal health, and/or a multivitamin to support overall health. In another example, instead of containing coupons for additional products, the kit can contain samples and/or full size products.

The biomarker test can be any test that can effectively test the biomarker. In one example, the biomarker test can be a blood test. In one example, the consumer can fast for 8 to 12 hours before the blood test. In another example, the consumer does not have to fast before the blood test. The blood test can use any quantity of blood, however, in certain examples the blood test only uses a finger prick of blood. In another example, the biomarker test can test and analyze things such as the consumer's sweat, body temperature, heart rate, breath, salvia, urine, tears, and skin chemistry.

In one example, the user can go to a walk-in healthcare clinic to receive their biomarker test, for instance the Take Care Clinic at Walgreens® or the Minute Clinic® at CVS® or an urgent care clinic. In some instances, a mobile clinic may also conduct the biomarker tests. In one example, the consumer does not schedule an appointment before going to receive the biomarker test. The consumer can also get a test performed at a doctor's office or another resource where they an appointment is required. A healthcare professional such as a doctor, nurse including a nurse practitioner, physician's assistant, pharmacist, or laboratory technician can perform the biomarker test(s). In some examples, the kit contains a voucher or coupon for a biomarker test at a particular location or brand of healthcare clinics, for instance the kit could contain a voucher or coupon for a specific pharmacy or all pharmacy's owned by a particular company. In another example, the consumer receives the biomarker test at work or another central meeting location. In another example, the consumer can receive the test at work, either through an already established health care clinic that can conduct a biomarker test or a special event.

In another example, a consumer could bring in the product they are already using and show it to a pharmacist or other health practitioner or contact the pharmacy or store with a UPC # (Universal Product Code) or other information to have a test kit mailed to them for to obtain baseline. After a certain amount of time another test kit could be sent to obtain results. A follow up call could be made to assist the consumer in understanding the results or it could be suggested they talk to a local health practitioner such as their primary care physician or local pharmacist.

In another example, the pharmacy or store where consumers purchase their VMS product could provide additional information related to either the VMS product, other VMS products, or additional health and wellness resources available at the pharmacy or store. In some examples, stores and pharmacies with health clinics could see an overall increase in the use of the health clinic after offering unseen benefit tests. In another example, stores and pharmacies could see an overall increase in sales of VMS products.

In some examples, the consumer can receive the results soon after the test is conducted. For instance, at certain healthcare clinics, the consumer could receive the results within an hour of taking the test, in other examples within thirty minutes, and in other examples within fifteen minutes. In some examples, the consumers can receive the test results while she is shopping for other items.

In some examples the biomarker test can be performed by the user at home or on-the-go. In one example, the user can conduct the test at home and send the results to a laboratory. In another example, the user can get the results for themselves. For instance, a device or application, such as an application on a Smartphone or computer. In another example, the kit can provide something that analyzes the results that can be easily analyzed by the consumer. In another example, the Smartphone or computer application that analyzes the biomarker can also help the consumer understand the results. The application can also remind the consumer to repurchase VMS products and/or can recommend additional VMS products the consumer may be interested in. In another example, the biomarker test can be included in the kit. In yet another example, a voucher or coupon for the test can be included in the kit. In another example, the test can be mailed to the consumer.

In one example, the consumer can perform the biomarker test twice while using the unseen benefit indicator. The biomarker test can be performed before taking the VMS product and then after a predetermined amount of time, in this case the user can compare her final result with the baseline.

In another example, the user can get multiple biomarker tests during the time period she is taking the VMS product. For instance, the consumer may take three biomarker tests, for instance, one test as a baseline test, one test at an approximate midway point, and then the final biomarker test. This would allow the user to see the progress. This could be especially helpful if the effects of the VMS product tend to be cumulative and further improvement in the biomarker can occur over time.

In another example, the user may get biomarker readings as frequently as desired. For instance, the biomarker readings could come from something that the user was wearing, such as a sensor that analyzes biomarkers in a user's sweat, a contact that can measure biomarkers in tears, or an instrument the user can carry with her that can non-invasively analyze biomarkers, such as something that analyzes breath or saliva, or clothing with sensors embedded in it. The user could track the biomarkers throughout the day and over the course of the use of the product.

Non-limiting examples of vitamins, minerals, and supplements can include probiotics, prebiotics, vitamin A, vitamin B1 (Thiamine), vitamin B2 (riboflavin), vitamin B3 (niacin), vitamin B6, vitamin B12, vitamin C, vitamin D, vitamin E, folate, vitamin K, calcium, chromium, copper, fluoride, iodine, iron, magnesium, manganese, molybdenum, phosphorus, potassium, selenium, zinc, pantothenic acid, pyridoxine, biotin, omega-3 fatty acids (eicosapentaenoic acid (EPA), Docosahexaenoic Acid (DHA), Octadecatetraenoic Acid; Docosapentaenoic Acid; Linolenic Acid; Heneicosapentaenoic Acid; Eicosatrienoic Acid), omega-6 fatty acids (Linoleic Acid; Arachidonic Acid; Eicosadienoic Acid), omega-5 & 7 fatty Acids (Myristoleic Acid; Palmitoleic Acid; Vaccenic Acid), omega-9 Fatty Acids (Oleic Acid; Gadoleic Acid; Erucic Acid; Nervonic Acid), astaxanthin, oregano, rosemary, turmeric, ginger, holy basil, green tea, hu zhang, Chinese goldthread, barberry, organic oregano, Chinese skullcap, saw palmetto extract, pumpkin seed oil, nettle extract, schizandra, maca, chamomile, fenugreek, aloe, peppermint, coriander, cardamom, artichoke, elder, eleuthero, astragalus, hawthorne, lemon balm, Indian Pink (Silene californica), Morning Glory (Ipomoea purpurea), Penstemon (Penstemon davidsonii), Self-Heal (Prunella vulgaris), Zinnia (Zinnia elegans), cinnamon, garlic, clove, grape seed, green tea, white tea, black tea, Camellia sinensis, rhodiola, sea buckthorn, St. John's Wort, black currant, evening primrose, schizandra, chaste tree, parsley, inulin, oligofructose, psyllium, guar gum, pectin, chitosan, cellulose, methylcellulose, beta-gucans, polydextrose, resistant dextrins, fructooligosaccharides, acacia fibers, melatonin, and combinations thereof.

In one example, the VMS product can include one or more vitamins, minerals, or supplements and can be taken for one or more health benefits. Non-limiting examples of health benefits can include appetite control, helps you feel less hungry between meals, supporting restful sleep, supporting liver health, supporting hair, skin, and/or nail health, supporting energy, supporting positive mood, maintaining joint and/or cartilage health, supporting digestive health and/or digestive function, supporting inner wellbeing, whole-body healthy inflammation response, prostate health, heart and/or cardiovascular health, nourishing moving joints and/or cartilage, supporting men's health, supporting women's health, supporting health of men and/or women over 40, soothing stress support, maintaining or supporting healthy blood sugar levels, promoting healthy weight management, supporting a body's natural ability to produce energy, promoting balanced energy and endurance, supporting healthy aging, supporting longevity, supporting vitality, supporting cellular protection, supporting healthy mood and spirit, supporting alertness and focus, supporting cognitive health, pain relief, supporting muscular health, supporting eye health, supporting prenatal health and fetal development, protecting against oxidative stress, free-radical scavenger, supporting normal respiratory function, supporting stamina, endurance, and well-being under conditions of exertion and sleep disruption, shifting the body's stress response, uplifting mood, providing positive emotional health support, holistic bone nourishment, promoting healthy hormonal balance, promoting detoxification, promoting antioxidant responses, daily immune support, lowering total cholesterol, lowering bad/LDL cholesterol, and combinations thereof.

In one example, the vitamins, minerals, and supplements can be naturally sourced. For instance, omega fatty acids can be sourced from fish, such as Wild Alaskan Salmon and in another example calcium can come from red marine algae (lithothamnion). In another example, the vitamins, minerals and supplements can be synthesized. In another example the vitamins, minerals and supplements can be supercritical, hydroethanolic or aqueous extracts. In another example, the vitamins, minerals, and supplements can be fermented. In another example, the vitamins, minerals, and supplements can be whole-food complexed vitamins In another example the VMS supplement can comply with the non-GMO Project Standard. In another example the VMS supplement can be organic.

In another example, the unseen benefit indicator can be used in an area where people would like to improve their health. This could include a city, neighborhood, workplace, school, social group, or the like. For instance, a workplace may be looking to lower stress and a group of employees could get baseline readings of a biomarker for stress, such as cortisol, and then begin a VMS regimen with a vitamin intended to reduce stress, such as a B vitamins, a multi-vitamin containing B vitamins and/or vitamin C that helps to maintain the structure of the adrenal glands for a healthy stress response and/or a VMS product that can be used to help with sleep. In addition, another regimen such as healthy eating and/or an exercise program can also be implemented to further lower stress and/or improve health or wellness.

In another example, people who are at risk of heart attack and/or have high cholesterol are targeted to use the unseen benefit indicator, with the hope that using this system will prevent them from having to take additional prescription medications to control cholesterol, high blood pressure and other problems. These consumers may take fish oil and/or psyllium fiber.

In another example, a service could be offered to insurance agencies that would help them target customers who are at risk for long-term health issues, including heart disease. The insurance agency could contact these consumers with kits and encourage them, possibly via a monetary rewards program or other program, to closely track their bio-markers to avoid long-term issues.

In another example, the baseline test may look at a user's skin and in one example the user's skin could be scanned. Then, a product can be used that could improve skin color, texture, and/or provide anti-aging benefits. It can be very difficult to tell if a product is working to improve skin color, texture, and the test can give an indication of whether or not the product is working. This can be particularly helpful for a product, such as an anti-aging product, where the results are not realized for a long time. A similar methodology could be used in oral care for teeth whitening products.

In another example, the unseen benefit kit can be intended to help consumer's sleep. In one example, the kit can contain a supplement to help a user sleep, such as melatonin, a relaxing tea, and/or an over-the-counter medicine, such as diphenhydramine, or a prescription sleep aid. The user can get their baseline. In one example, the baseline can be done with a sensor. The sensor can determine what stages of sleep the person is in, for instance light sleep, deep sleep, REMS (rapid eye movement sleep), awake, etc. The baseline measurements can be taken over one night or over a series of nights, in order to get an accurate and complete baseline value. In another example, the test can track the user's movement while she sleeps. Then the user can take the sleeping compositions and/or combination of sleeping compositions. The test can be repeated. Consumers may want to repeat the test the first night that they are on the regimen; however, it can be recommended that they continue the regimen for several nights or a few weeks in order to determine if it is efficacious for them. In one example, the sensor can communicate with a Smartphone or computer application and can help the user visualize and track their progress. The consumer can then decide to continue with the regimen that helps them sleep. In another example, a biomarker test as described above can be used that measures biomarkers related to stress, such as cortisol, to see if the products are helping the consumer.

In another example, the unseen benefit kit can help a user determine if their cold/flu medication is working, in particular multi-symptom relief cold/flu medication that can include a cough suppressant, nasal decongestant, pain reliever and/or an expectorant. In one example, the baseline test can measure the user's temperature by applying a sensor to the user's skin and/or oral mucosa. After taking the medication, the user can apply the sensor again to take her temperature. This test can be repeated as often as desired and can be repeated every hour, every two hours, every three hours, every four hours, every five hours, every six hours, every seven hours, or every eight hours. In another example, the test can be performed in real time or as needed. In a similar fashion, a sensor can indicate whether or not there is an improvement in cough.

In one example, the user can buy the kit at a store and have the biomarker test performed at the same store. In another example, the user can buy the kit at a store and/or online and can have the biomarker test performed at a different retailer/store. In one example, the kit can contain a voucher or coupon for a biomarker test at a specific store. In another example, the kit can contain the biomarker test and the user can conduct the test at home. In another example, the biomarker test can be purchased separately. In another example, the biomarker test can be the same brand as the VMS product and/or can be owned by the same parent company. In another example, the biomarker test can be an application that can be downloaded on the internet and the VMS product can instruct the user how to obtain the application. In another example, the biomarker test can be measured through wearable technology that includes a sensor. In another example, the biomarker test can be in a user's bed or pillow. In another example, the biomarker test can measure pressure points. In another example, the consumer goes online to learn how to use the test. In another example, the kit and/or the test and/or the supplement are purchased online.

In another example, after using the unseen benefit indicator consumers can subscribe to receive VMS products regularly. In some examples, the consumers may receive additional benefits and/or discounts if they use social media or other formats to discuss the benefits of the test.

In another example, the unseen benefit indicator can be used with over-the-counter medications either instead of or in combination with VMS products. Non-limiting examples of over-the-counter medications can include guaifenesin, ambroxol, bromhexine, chlorpheniramine, desloratadine, levocetirizine, diphenhydramine, doxylamine succinate, triprolidine, clemastine, pheniramine, brompheniramine, dexbrompheniramine, loratadine, cetirizine and fexofenadine, amlexanox, alkylamine derivatives, cromolyn, acrivastine, ibudilast, bamipine, ketotifen, nedocromil, omalizumab, dimethindene, oxatomide, pemirolast, pyrrobutamine, pentigetide, thenaldine, picumast, tolpropamine, ramatroban, repirinast, suplatast tosylate aminoalkylethers, tazanolast, bromodiphenhydramine, tranilast, carbinoxamine, traxanox, chlorphenoxamine, diphenylpyaline, embramine, p-methyldiphenhydramine, moxastine, orphenadrine, phenyltoloxamine, setastine, ethylenediamine derivatives, chloropyramine, chlorothen, methapyrilene, pyrilamine, talastine, thenyldiamine, thonzylamine hydrochloride, tripelennamine, piperazines, chlorcyclizine, clocinizine, homochlorcyclizine, hydroxyzine, tricyclics, phenothiazines, mequitazine, promethazine, thiazinamium methylsulfate, azatadine, cyproheptadine, deptropine, desloratadine, isothipendyl, olopatadine, rupatadine, antazoline, astemizole, azelastine, bepotastine, clemizole, ebastine, emedastine, epinastine, levocabastine, mebhydroline, mizolastine, phenindamine, terfenadine, tritoqualine, phenylephrine (PE), pseudophedrine (PSE), dextromethorphan, codeine, chlophedianol, levodropropizine, acetaminophen, ibuprofen, ketoprofen, diclofenac, naproxen, aspirin, caffeine, bismuth subsalicylate, simethicone, diphenhydramine, ascimetidine, ranitidine, phosphorated carbohydrate, omeprazole, lansoprazole, calcium carbonate, aluminum hydroxide, magnesium hydroxide, and combinations thereof.

In another example, the unseen benefit indicator can be used with functional foods, which can include drinks, snack bars, and other edible items. In some examples, functional foods can contain VMS products, like fiber, probiotics, and/or other vitamins and minerals. In another example, functional foods can contain protein. In another example, function foods can contain electrolytes.

In another example, the unseen benefit indicator can be used with skin products and in another example with oral care products.

### Combinations

A. An unseen benefit indicator kit comprising:
a. a vitamin, mineral, supplement product;
b. a first biomarker test or a voucher for a first biomarker test or a coupon for a first biomarker test;
c. a final biomarker test or a voucher for a final biomarker test or a coupon for a final biomarker test;
   wherein the kit provides an indicator system that allows a user to see a health benefit while taking the vitamin, mineral, supplement product.

B. The unseen benefit indicator of paragraph A wherein the first biomarker test and the final biomarker test are blood tests and test for triglycerides, low-density lipoprotein, high-density lipoprotein, total cholesterol and combinations thereof.

C. The unseen benefit indicator kit of any one of the preceding paragraphs A-B wherein the vitamin, mineral, supplement product is fish oil.

D. The unseen benefit indicator kit of any one of the preceding paragraphs A-C wherein the vitamin, mineral, supplement product is psyllium.

E. The unseen benefit indicator kit of any one of the preceding paragraphs A-D wherein the first biomarker test and the final biomarker test are blood tests and test for inflammation markers selected from the group consisting of NFkB, PGE2, COX1, COX2, NRF-2, LTB4, SLOX, 12LOX, CRP, Interleukins, TNFα, INF and combinations thereof.

F. The unseen benefit indicator kit of any one of the preceding paragraphs A-E wherein the health benefit is a change in the biomarker in the positive direction.

G. The unseen benefit indicator kit of any one of the preceding paragraphs A-F wherein the kit contains at least a two week supply of the vitamin, mineral, supplement.

H. The unseen benefit indicator kit of any one of the preceding paragraphs A-G further comprising coupons or vouchers for additional vitamin, mineral, supplement products.

I. The unseen benefit indicator kit of any one of the preceding paragraphs A-H wherein the health benefit cannot be seen or experienced by the user without biomarker tests.

J. A method for seeing an unseen benefit comprising:
a. obtaining the unseen benefit indicator kit of paragraph A;
b. taking the first biomarker test and receiving a first biomarker test result;
c. consuming the vitamin, mineral, supplement product;
d. taking the final biomarker test and receiving a final biomarker test result;
e. comparing the first biomarker test result and the final biomarker test result to see the unseen benefit.

K. The method of paragraph J further comprising recording a user's reaction when she compares the first biomarker test result with the second biomarker test result and using it in an advertisement.

L. The method of any one of the preceding paragraphs J-K further comprising deciding whether or not to continue consuming the vitamin, mineral, supplement product after comparing the first and final biomarker test results.

M. The method of any one of the preceding paragraph J-L wherein the first biomarker test or the final biomarker test are performed at a healthcare clinic.

N. The method of any one of the preceding paragraph J-M wherein the first biomarker test or the final biomarker test are performed at a user's home.

O. The method of any one of the preceding paragraph J-N where the results are reviewed with a healthcare professional.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An unseen benefit indicator kit comprising:
a. a vitamin, mineral, supplement product;
b. a first biomarker test or a voucher for a first biomarker test or a coupon for a first biomarker test;
c. a final biomarker test or a voucher for a final biomarker test or a coupon for a final biomarker test;
wherein the kit provides an indicator system that allows a user to see a health benefit while taking the vitamin, mineral, supplement product.

2. The unseen benefit indicator of claim 1 wherein the first biomarker test and the final biomarker test are blood tests and test for triglycerides, low-density lipoprotein, high-density lipoprotein, total cholesterol and combinations thereof.

3. The unseen benefit indicator kit of claim 2 wherein the vitamin, mineral, supplement product is fish oil.

4. The unseen benefit indicator kit of claim 2 wherein the vitamin, mineral, supplement product is psyllium.

5. The unseen benefit indicator kit of claim 1 wherein the vitamin, mineral, supplement product is an herbal supplement.

6. The unseen benefit indicator kit of claim 1 wherein the vitamin, mineral, supplement product is a probiotic.

7. The unseen benefit indicator kit of claim 1 wherein the first biomarker test and the final biomarker test are blood tests and test for inflammation markers selected from the group consisting of NFkB, PGE2, COX1, COX2, NRF-2, LTB4, SLOX, 12LOX, CRP, Interleukins, TNFα, INF and combinations thereof.

8. The unseen benefit indicator kit of claim 1 wherein the first biomarker test and the final biomarker test are blood tests and test for ketones.

9. The unseen benefit indicator kit of claim 1 wherein the health benefit is a change in the biomarker in the positive direction.

10. The unseen benefit indicator kit of claim 1 wherein the kit contains at least a two week supply of the vitamin, mineral, supplement.

11. The unseen benefit indicator kit of claim 1 wherein the kit contains at least a four week supply of the vitamin, mineral, supplement.

12. The unseen benefit indicator kit of claim 1 further comprising coupons or vouchers for additional vitamin, mineral, supplement products.

13. The unseen benefit indicator kit of claim 1 wherein the health benefit cannot be seen or experienced by the user without biomarker tests.

14. A method for seeing an unseen benefit comprising:
a. taking a first biomarker test and receiving a first biomarker test result;
b. consuming a vitamin, mineral, supplement product;
c. taking a final biomarker test and receiving a final biomarker test result;
d. comparing the first biomarker test result and the final biomarker test result to see an unseen benefit.

15. The method of claim 14 further comprising recording a user's reaction when she compares the first biomarker test result with the second biomarker test result and using it in an advertisement.

16. The method of claim 15 wherein the advertisement is a video advertisement.

17. The method of claim 14 further comprising deciding whether or not to continue consuming the vitamin, mineral, supplement product after comparing the first and final biomarker test results.

18. The method of claim 14 wherein the first biomarker test or the final biomarker test are performed at a healthcare clinic.

19. The method of claim 14 wherein the first biomarker test or the final biomarker test are performed at a user's home.

20. The method of claim 14 where the results are reviewed with a health care professional.
